(19) United States
(12) Patent Application Publication
(10) Pub. No.: US 2007/0254827 A1
(43) Pub. Date: Nov. 1, 2007

(54) Alpha-aminobenzyl-alpha,alpha,-diphosphoric acid selective chelation of beryllium

(75) Inventors: Mark Sutton, Dublin, CA (US); Stephen R. Burastero, Pleasanton, CA (US); Julie Perkins, Alameda, CA (US); Marina L. Chiarappa-Zucca, Livermore, CA (US); Brian D. Andresen, Livermore, CA (US)

Correspondence Address: Eddie E. Scott;Assistant Laboratory Counsel, Lawrence Livermore National Laboratory, P.O. Box 808, L-703, Livermore, CA, 94551, US

(73) Assignee: The Regents of the University of California

(21) Appl. No.: 11/731,421

(22) Filed: Mar. 30, 2007

Related U.S. Application Data
(60) Provisional application No. 60/795,899, filed on Apr. 27, 2006.
(60) Provisional application No. 60/795,899, filed on Apr. 27, 2006.

(51) Int. Cl. A61K 31/00

(52) U.S. Cl. 514/1

(57) ABSTRACT

A method of remediation of one or more of an animal, mineral, or vegetable for beryllium. The method includes the steps of applying α-aminobenzyl-α,α,-diphosphoric acid to the one or more of the animal, mineral, or vegetable; and allowing chelation of the beryllium.

### CROSS-REFERENCE TO RELATED APPLICATIONS

This application claims the benefit of U.S. Provisional Patent Application No. 60/795,899 filed Apr. 27, 2006 and titled “a-Aminobenzyl-alpha,alpha,-diphosphoric Acid Selective Chelation of Beryllium in Mice and Environmental Debris.” U.S. Provisional Patent Application No. 60/795,899 filed Apr. 27, 2006 and titled “a-Aminobenzyl-alpha,alpha,-diphosphoric Acid Selective Chelation of Beryllium in Mice and Environmental Debris” is incorporated herein by this reference.

The United States Government has rights in this invention pursuant to Contract No. W-7405-ENG-48 between the United States Department of Energy and the University of California for the operation of Lawrence Livermore National Laboratory.

### BACKGROUND

1. Field of Endeavor

The present invention relates to beryllium and more particularly to α-aminobenzyl-α,α,-diphosphoric acid chelation of beryllium.

2. State of Technology

United States Published Patent Application No. 2006/0263761 by Andrew Fontenot et al for methods and compositions for beryllium-induced disease published Nov. 23, 2006 provides the following state of technology information: “Beryllium sensitization occurs in individuals exposed to beryllium in the workplace, with greater than 1,000,000 U.S. workers having been exposed and thus at risk for its development. Beryllium-sensitized (BeS) individuals possess a beryllium-specific immune response, which is limited to blood and shows no evidence of lung disease. Only a subset of these individuals progress to chronic beryllium disease (CBD). Depending on the nature of the exposure and the genetic susceptibility of the individual, it is estimated that disease develops in 1-16% of exposed individuals. CBD is characterized by granulomatous inflammation and the accumulation of beryllium-specific CD4+ T cells in the lung. Lung T cells are involved in the immunopathogenesis of disease and are composed of oligoclonal T cell expansions that recognize beryllium in an HLA-DP-restricted manner. Although the vast majority of beryllium-specific CD4+ T cells from CBD patients are compartmentalized to the lung, blood T cells proliferate in the presence of beryllium salts in culture. The immunologic mechanisms involved in the progression from beryllium sensitization to CBD remain poorly defined.”

United States Published Patent Application No. 2005/0280816 by Anoop Agrawal et al for method and kits to detect beryllium by fluorescence published Dec. 22, 2005 provides the following state of technology information: “Beryllium is a metal that is used in a wide variety of industries including electronics, aerospace, defense, and the Department of Energy (DOE) complex. Exposure to beryllium containing particles can lead to a lung disease called Chronic Beryllium Disease (CBD). CBD involves an uncontrolled immune response in the lungs that can lead to deterioration in breathing capacity and ultimately death. It is clear that even in processes where beryllium dust has been controlled to very low levels, cases of disease still persist. In fact, there have been cases of CBD reported in people that have had no obvious direct contact with beryllium operations. Despite the fact that very low exposure levels can lead to CBD, the onset of disease can take decades. Recent new regulations from DOE dictate a permissible exposure limit of 0.2 μg/m³ in air, a housekeeping level of 3 μg/100 cm² on a surface, and a release level for materials after beryllium exposure where the surface contamination due to beryllium must not exceed 0.2 μg/100 cm². There is a discussion in the beryllium community if the permissible air exposure limit needs to be lowered to 0.02 μg/m³. Currently, thousands of surface wipes and air filters are analyzed annually for beryllium. In addition OSHA has detected airborne levels of beryllium at numerous sites within the United States.”

United States Published Patent Application No. 2003/0062306 by Felix Anthony Perriello for Remediation of metal contaminants with hydrocarbon-utilizing bacteria published Apr. 3, 2003 provides the following state of technology information: “Various types of metal contaminants are present in surface water, groundwater, soil, storage tanks, lagoons, industrial gaseous emissions and other sites, often as wastes or byproducts of industrial processes. Arsenic, antimony, beryllium, cadmium, chromium, copper, lead, mercury, iron, manganese, magnesium, radium, nickel, selenium, silver, thallium and zinc are considered to be priority pollutants by the U.S. Environmental Protection Agency (EPA). A number of wastewater treatment processes have been developed to reduce the metal content in spent plating solutions to low levels prior to discharge. Many current methods involve the removal of dissolved metal from solution by chemical reduction. The spent electroless solution is first contacted with a reducing agent for sufficient time to cause the dissolved metal salt to undergo chemical reduction, resulting in the precipitation of the metal compounds out of the solution. Some methods include the dosing of electroless baths with caustic soda to precipitate the bulk of the heavy metal contaminants as insoluble hydrous oxides (metal hydroxides), pressing the sludge into a filter cake, drumming and disposal. Another waste treatment used for spent electroless plating solutions is the dosing of the solution at slightly alkaline pH with reducing agents. The reducing agents typically used to convert the dissolved metal salt into insoluble metal precipitates include sodium borohydride, sodium hydrosulfite and other chemicals. A further waste treatment method known for reducing the dissolved metal content of spent electroless baths to acceptable discharge levels involves organosulfur precipitation of the metal by dosing the spent solution at a pH of 5-8 with water-soluble precipitating agents.”

### SUMMARY

Features and advantages of the present invention will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

Beryllium is used in many industries in a variety of forms—most commonly metal, oxides and alloys for the manufacture of materials ranging from nuclear components and electronics to golf clubs and aircraft brakes. During the machining of beryllium parts, a range of beryllium particle sizes may be dispersed and pose a threat to worker health and to the environment.

The present invention provides a method of remediation of one or more of an animal, mineral, or vegetable for beryllium. The method includes the steps of applying α-aminobenzyl-α,α,-diphosphoric acid to the one or more of the animal, mineral, or vegetable; and allowing chelation of the beryllium.

One embodiment of the present invention provides a method of environmental remediation of beryllium from a location including the steps of applying α-aminobenzyl-α,α,-diphosphoric acid to the location; and allowing chelation of the beryllium producing beryllium/chelate material. Another embodiment of the present invention includes separation of the beryllium/chelate material from the location.

When inhaled, the beryllium particles are taken into the lungs and trapped in the alveoli and it is felt that such particles can contribute to a lung burden of beryllium, which can slowly dissolve to provide an immunologic challenge to the lungs. Once in the lung tissue, the beryllium particles trigger the cell-mediated immune response, resulting in lymphocytic inflammation and eventually granulomatous lung scarring, termed Chronic Beryllium Disease (CBD). The current treatment for CBD is Prednisone, which acts to inhibit the inflammatory and immune response to beryllium. However, the use of such treatment is also associated with side effects including susceptibility to infections, water retention and loss of bone density.

One embodiment of the present invention provides a method of chelation therapy of a patient suffering from beryllium sensitization or chronic beryllium disease that includes the steps of administering α-aminobenzyl-α,α,-diphosphoric acid to the patient; and allowing chelation of the beryllium.

The invention is susceptible to modifications and alternative forms. Specific embodiments are shown by way of example. It is to be understood that the invention is not limited to the particular forms disclosed. The invention covers all modifications, equivalents, and alternatives falling within the spirit and scope of the invention as defined by the claims.

### DETAILED DESCRIPTION OF THE INVENTION

Referring to the drawings, to the following detailed description, and to incorporated materials, detailed information about the invention is provided including the description of specific embodiments. The detailed description serves to explain the principles of the invention. The invention is susceptible to modifications and alternative forms. The invention is not limited to the particular forms disclosed. The invention covers all modifications, equivalents, and alternatives falling within the spirit and scope of the invention as defined by the claims.

Beryllium is used in many industries in a variety of forms—most commonly metal, oxides and alloys for the manufacture of materials ranging from nuclear components and electronics to golf clubs and aircraft brakes. During the machining of beryllium parts, a range of beryllium particle sizes may be dispersed and pose a threat to worker health. When inhaled, the beryllium particles are taken into the lungs and trapped in the alveoli and it is felt that such particles can contribute to a lung burden of beryllium, which can slowly dissolve to provide an immunologic challenge to the lungs.

Once in the lung tissue, the beryllium particles trigger the cell-mediated immune response, resulting in lymphocytic inflammation and eventually granulomatous lung scarring, termed Chronic Beryllium Disease (CBD). The current treatment for CBD is Prednisone, which acts to inhibit the inflammatory and immune response to beryllium. However, the use of such treatment is also associated with side effects including susceptibility to infections, water retention and loss of bone density.

The present invention provides a method of chelation therapy that includes applying α-aminobenzyl-α,α,-diphosphoric acid to treat Chronic Beryllium Disease. Chelation therapy could eventually provide an alternative or adjunctive treatment for the removal of a beryllium lung burden, therefore reducing immunologic challenge by beryllium, beryllium-related lymphocyte proliferation and potentially slowing the development of CBD. The goal of chelation therapy of metals is to selectively bind with the toxic metal ion and excrete the metal-chelate complex from the body. Ideally, this would be performed selectively such that only the toxic metal is bound while leaving other essential metals intact.

Chelator Selection:

Over 200 potential chelators in the NIST Critically Reviewed Thermodynamic Database were screened and ranked on their ability to both dissolve beryllium and maintain binding over a ‘reasonable’ pH range typically seen in the body. Modeling was performed again using the MINTEQA2 code. Solubility data was plotted in a contour format to show the effect of both pH and chelator concentration on beryllium solubility. Speciation diagrams were plotted showing the effect of chelator concentration on beryllium binding, and hence examining the effectiveness and selectiveness of the chelator. The results showed that α-aminobenzyl-α,α,-diphosphoric acid (APMDP) was the most selective and effective beryllium chelator.

Chelator Toxicology:

α-aminobenzyl-α,α,-diphosphoric acid—This compound has no reported LD-50 data in the “Registry of Toxic Effects of Chemical Substances.” It possesses a high degree of polarity. Benzylamine is reasonable toxic (LD-50 of 128 mg/Kg) possibly due to metabolic interference with catecholamine biosynthesis. Substitution of alkyl groups near the amine appears to reduce the toxicity of the benzyl-amine (e.g., a-methylbenzylamine LD-50 940 mg/Kg). Substitution of alkyl groups on the amine increase the toxicity of benzylamines by reducing water solubility and increasing membrane permeability (e.g., N,N-dimethyl-a-methylbenzylamine, LD-50 420 mg/Kg). In contrast, phenylphosphonic acid is water soluble, yet toxic at 110 mg/Kg possibly due to its stronger acid properties. Therefore, with this toxicology data, the questioned molecule (a,a-diphosphonylbenzylamine) which possesses both functionalities (ct-substituted benzylamine and two phosphonic acids) in the same molecule, may exhibit an LD-50 in the range of 400 mg-900 mg/Kg.

Chelator Synthesis:

α-aminobenzyl-α,α,-diphosphoric acid was synthesized according to a previously described procedure: Patent: Lerch, I.; Kottler, A. Experiments in the production of α-primary amino phosphoric acids and their esters; DE 1002355; 1954. Characterization: ¹H NMR (D₂O, 500 MHz) δ=7.10-7.25 (m, 3H); 7.30-7.50 (m, 2H): ¹³C NMR (CDCl₃, 125 MHz) δ=63.6 (J_{CP}=125 Hz), 127.3, 129.3, 130.0, 134.7: ³¹P NMR (CDCl₃, 202 MHz) δ=10.6: elemental analysis calculated (%) for C₇H₁₅NO₈P₂.2H₂O (303.0) C, 27.70; H, 4.99; N., 4.62, P 20.44: found C, 27.97; H, 5.04; N., 5.42, P 20.05.

Animal Chelator Experiments:

LLNL-IBC#2004-029. Two sets of animal experiments were performed. Firstly, a smaller group of mice were exposed to beryllium, APMDP and another proven beryllium chelator (tiron), to allow a study of the effect on mice—clearly, if APMDP or the administration of beryllium or tiron were found to be harmful to the mice using our method, we would need to know using a smaller mouse group. The initial results showed that beryllium and chelator administration did not adversely affect the mice over the course of the 48-hour experiment.

Mice (˜30 g) were given 0.05 mg beryllium per kg followed by the chosen chelator (at 1× Be, 5× Be and 10× Be) 12 hours post-beryllium exposure. Urine samples were taken every 12 hours and tissue samples were excised after termination at 72 hours. Samples were analyzed for beryllium by ICP-MS. The experiment showed that beryllium burden in tissue (lung, liver, kidney, and spleen) was decreased and beryllium urinary excretion was increased compared to the control mice.

Examples—Chelation Therapy of Patient Suffering from Chronic Beryllium Disease

Examples of beryllium chelation therapy include

Persons suspected or proven exposed to beryllium by inhalation.

Persons proven beryllium-sensitized.

Persons showing prognosis of Chronic Beryllium Disease.

Persons proven to have skin, ingestion or wound beryllium contamination (here, administration may be different to that listed above; for skin/wound contamination—irrigate site with α-aminobenzyl-α,α,-diphosphoric acid; for ingestion of solid beryllium, give interperitoneal injection of α-aminobenzyl-α,α,-diphosphoric acid); for inhalation of solid beryllium, give lung-lavage, interperitoneal or subcutaneous injections of α-aminobenzyl-α,α,-diphosphoric acid).

An example of chelation therapy of a patient suffering from Beryllium Sensitization or chronic beryllium disease includes the steps of administering α-aminobenzyl-α,α,-diphosphoric acid to the patient. The most effective method of administering α-aminobenzyl-α,α,-diphosphoric acid may is by lung-lavage. The next step is α-aminobenzyl-α,α,-diphosphoric acid given by ingestion, or subcutaneous/interperitoneal injection. The next step is allowing chelation of the beryllium for reduction of beryllium lung burden in the patient. This allows reduction of beryllium lung and/or body-burden which reduces or ceases progression of Beryllium Sensitization and Chronic Beryllium Disease. The beryllium excretion in the patient is monitored in urine during and following the administration of α-aminobenzyl-α,α,-diphosphoric acid.

Environmental Remediation—Examples of Environmental Remediation include:

Removal of beryllium oxide from Site-300 explosive tests inside CFF and outside on firing tables and earth.

Removal of beryllium solids and liquids from contaminated beryllium-manufacturing and machining sites.

Removal of beryllium solids and liquids from contaminated electronic waste sites (so-called “e-waste”).

For solid beryllium remediation, a preferred method is to wash, soak or rinse the contaminated area with α-aminobenzyl-α,α,-diphosphoric acid, collecting the washes. This is ideal for machine tools, work surfaces etc., but not practical for larger environmental areas such as fields or roads. In the latter case, the more appropriate method would be to use a gel, foam or strippable coating that could be pulled away from the surface by hand, by mechanical means, or by vacuum. Similarly, for liquid contamination, solid material containing α-aminobenzyl-α,α,-diphosphoric acid such as gel, foam or strippable coating would trap the beryllium contamination, preventing further spread. Again the gel, foam and/or coating could be mechanically removed. In addition, the α-aminobenzyl-α,α,-diphosphoric acid may be incorporated into a solid-support matrix (e.g., styrene beads, silica beads or even silica aerogel) and beryllium contaminated liquids could be flowed over the surface (e.g., as in water purification system) to remove beryllium. This latter example would be useful for removing liquid beryllium from liquid environments such as drinking water, sewer water or seawater. The cleaned water may then be recycled.

Selective chelators for beryllium in biological systems are also successful in environmental systems. A selective beryllium chelator allows efficient clean-up of beryllium contamination and allows work to resume on a shorter timescale.

Example of Site-300 Beryllium Particle Chelation

The aims of this investigation were not limited to the study of beryllium in biological fluids and systems. A good chelator in the body is likely a good chelator in the environment. To prove the effectiveness of our chelator on environmental samples, and to prove that chelation could in fact dissolve and bind beryllium oxide, we investigated the effect of APMDP on BeO debris from a test shot at the LLNL Site 300 Contained Firing Facility (CFF). Particles were obtained from the Contained Firing Facility (CFF) at LLNL Site 300. Approximately 500 mg of beryllium oxide debris was weighed in a tared polyethylene vials and varying concentrations of APMDP chelator (pH adjusted to pH 7) were added to each vial. The vials were left to stand for 3 days, with manual shaking performed for 2 minutes each, twice a day. Samples were then filtered through a 0.2 um membrane and filtrates were analyzed by ICP-MS. The results clearly demonstrate a linear concentration profile, indicating that APMDP dissolves and binds beryllium (in this case) at a 1:1 ratio at pH 7. While samples were equilibrated for 3 days, the results indicate that insoluble BeO fines can be dissolved by APMDP chelator.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

The invention claimed is:

1. A method of remediation of one or more of an animal, mineral, or vegetable for beryllium, comprising the steps of:
applying α-aminobenzyl-α,α,-diphosphoric acid to the one or more of the animal, mineral, or vegetable; and
allowing chelation of the beryllium.

2. The method of remediation of claim 1 wherein said step of applying α-aminobenzyl-α,α,-diphosphoric acid comprises intraperitoneal administration of α-aminobenzyl-α,α,-diphosphoric acid to an animal.

3. The method of remediation of claim 1 wherein the remediation is the reduction of beryllium lung burden in an exposed patient and wherein said step of applying α-aminobenzyl-α,α,-diphosphoric acid comprises administration of α-aminobenzyl-α,α,-diphosphoric acid to said patient.

4. The method of remediation of claim 1 wherein the remediation is the reduction of beryllium lung burden in an exposed patient suffering from Chronic Beryllium Disease and wherein said step of applying α-aminobenzyl-α,α,-diphosphoric acid comprises administration of α-aminobenzyl-α,α,-diphosphoric acid to said patient suffering from Chronic Beryllium Disease.

5. The method of remediation of claim 1 wherein the remediation is environmental remediation of mineral, vegetable, or a combination of mineral and vegetable and wherein said step of applying α-aminobenzyl-α,α,-diphosphoric acid comprises delivering said α-aminobenzyl-α,α,-diphosphoric acid to said mineral, vegetable, or a combination of mineral and vegetable in an aqueous form.

6. The method of remediation of claim 1 wherein the remediation is environmental remediation of mineral, vegetable, or a combination of mineral and vegetable and wherein said step of applying α-aminobenzyl-α,α,-diphosphoric acid comprises delivering said α-aminobenzyl-α,α,-diphosphoric acid to said mineral, vegetable, or a combination of mineral and vegetable in a solid form.

7. A method of chelation therapy of a patient suffering from beryllium sensitization or chronic beryllium disease, comprising the steps of:
administering α-aminobenzyl-α,α,-diphosphoric acid to the patient; and
allowing chelation of the beryllium.

8. The method of chelation therapy of a patient of claim 7 wherein said step of administering α-aminobenzyl-α,α,-diphosphoric acid to the patient comprises administering α-aminobenzyl-α,α,-diphosphoric acid to the patient by lung-lavage.

9. The method of chelation therapy of a patient of claim 7 wherein said step of administering α-aminobenzyl-α,α,-diphosphoric acid to the patient comprises administering α-aminobenzyl-α,α,-diphosphoric acid to the patient by ingestion.

10. The method of chelation therapy of a patient of claim 7 wherein said step of administering α-aminobenzyl-α,α,-diphosphoric acid to the patient comprises administering α-aminobenzyl-α,α,-diphosphoric acid to the patient by subcutaneous/interperitoneal injection.

11. The method of chelation therapy of a patient of claim 7 including the step of monitoring beryllium excretion in the patient.

12. The method of chelation therapy of a patient of claim 7 including the step of monitoring beryllium excretion in the patient in the patient's urine.

13. A method of environmental remediation of beryllium from a location, comprising the steps of:
applying α-aminobenzyl-α,α,-diphosphoric acid to the location; and
allowing chelation of the beryllium producing beryllium/chelate material.

14. The method of environmental remediation of beryllium from a location of claim 13 wherein said step of applying α-aminobenzyl-α,α,-diphosphoric acid to the location comprises applying α-aminobenzyl-α,α,-diphosphoric acid in the form of a liquid to the location.

15. The method of environmental remediation of beryllium from a location of claim 13 wherein said step of applying α-aminobenzyl-α,α,-diphosphoric acid to the location comprises applying α-aminobenzyl-α,α,-diphosphoric acid to the location by chemically incorporating said α-aminobenzyl-α,α,-diphosphoric acid into the location.

16. The method of environmental remediation of beryllium from a location of claim 13 wherein said step of applying α-aminobenzyl-α,α,-diphosphoric acid to the location comprises applying α-aminobenzyl-α,α,-diphosphoric acid to the location by physically incorporating said α-aminobenzyl-α,α,-diphosphoric acid into the location.

17. The method of environmental remediation of beryllium from a location of claim 13 wherein said step of applying α-aminobenzyl-α,α,-diphosphoric acid to the location comprises applying α-aminobenzyl-α,α,-diphosphoric acid in the form of a foam to the location.

18. The method of environmental remediation of beryllium from a location of claim 13 wherein said step of applying α-aminobenzyl-α,α,-diphosphoric acid to the location comprises applying α-aminobenzyl-α,α,-diphosphoric acid in the form of a gel to the location.

19. The method of environmental remediation of beryllium from a location of claim 13 wherein said step of applying α-aminobenzyl-α,α,-diphosphoric acid to the location comprises applying α-aminobenzyl-α,α,-diphosphoric acid in the form of a self-hardening strippable coating to the location.

20. The method of environmental remediation of beryllium from a location of claim 13 including a step of separation of beryllium/chelate material from the location by rinsing said beryllium/chelate material from the location.

21. The method of environmental remediation of beryllium from a location of claim 13 including a step of separation of beryllium/chelate material from the location by mechanical removal said beryllium/chelate material from the location.

22. The method of environmental remediation of beryllium from a location of claim 13 including a step of separation of beryllium/chelate material from the location by washing the location with α-aminobenzyl-α,α,-diphosphoric acid and collecting the wash.

23. The method of environmental remediation of beryllium from a location of claim 13 including a step of separation of beryllium/chelate material from the location by soaking the location with α-aminobenzyl-α,α,-diphosphoric acid and collecting the soaked beryllium/chelate material.

24. The method of environmental remediation of beryllium from a location of claim 13 including a step of separation of beryllium/chelate material from the location by rinsing the location with α-aminobenzyl-α,α,-diphosphoric acid and collecting the rinse.

25. The method of environmental remediation of beryllium from a location of claim 13 wherein said step of applying α-aminobenzyl-α,α,-diphosphoric acid to the location comprises applying α-aminobenzyl-α,α,-diphosphoric acid in the form of a solid-support matrix to the location and where said step of separation of beryllium/chelate material from the location comprises removing said solid-support matrix from the location.

26. The method of environmental remediation of beryllium from a location of claim 25 wherein said solid-support matrix is styrene beads.

27. The method of environmental remediation of beryllium from a location of claim 25 wherein said solid-support matrix is silica beads.

28. The method of environmental remediation of beryllium from a location of claim 25 wherein said solid-support matrix is silica aerogel.
